(19) United States
(12) Patent Application Publication
(10) Pub. No.: US 2022/0040346 A1
(43) Pub. Date: Feb. 10, 2022

(54) SYSTEM AND METHOD FOR SHOPPING CART DISINFECTION AND STERILIZATION USING ULTRAVIOLET LIGHT

(71) Applicant: Sterilization Station, LLC, Langley, WA (US)

(72) Inventors: Dan Phillips, Langley, WA (US); Tim Phillips, Freeland, WA (US)

(21) Appl. No.: 17/398,834

(22) Filed: Aug. 10, 2021

Related U.S. Application Data
(60) Provisional application No. 63/063,738, filed on Aug. 10, 2020.

(51) Int. Cl. A61L 2/10; G01P 13/00; G01G 19/02

(52) U.S. Cl. CPC A61L 2/10; G01P 13/00; G01G 19/02; A61L 2202/121; A61L 2202/122; A61L 2202/14; A61L 2202/11

(57) ABSTRACT

A system and method for sterilizing one or more high-touch implements, such as shopping carts, using ultraviolet (UV) light within a contained chamber for a specified period of time. In an embodiment, one or more shopping carts may be guided into a sterilization chamber through a front-side opening of an apparatus. Once the shopping cart is in place inside the sterilization chamber, a UV light process may be engaged whereby several UV lights will be turned on to flood the sterilization chamber with UV light. UV light has been shown to be effective at killing bacteria, viruses, mold, and other pathogens with exposure in as little as 5 seconds. The sterilization procedures results in sterilizing a shopping cart for next use as sterilized carts may be collected in a corral designated form newly sterilized shopping intended for next use.

### PRIORITY CLAIM

This application claims the benefit of U.S. Provisional Application No. 63/063,738, entitled “SYSTEM AND METHOD FOR SHOPPING CART STERILIZATION USING ULTRAVIOLET LIGHT”, filed Aug. 10, 2020, which is incorporated by reference in its entirety herein for all purposes.

### BACKGROUND

Shopping carts and other convenience items used by patrons of a store often come into heavy contact with many people during the course of use. In a single day, one shopping cart may used by as many as 30 people or more. Being an item of heavy use and repeated human contact, viruses and bacteria may collect and linger at high-touch areas, such as shopping carts handles. Lingering bacterial load and viral load can lead to a reduction in overall health of a population by spreading communicable disease.

However, bacteria and viruses may be reduced or eradicated with sterilization and/or exposure to disinfectants. A typical chemical-based disinfectant may be a substance or compound used to inactivate or destroy microorganisms on inert surfaces. Disinfection it is less effective than sterilization, which is an extreme physical or chemical process that kills all types of life. Sterilization is a physical or chemical process that destroys all living organisms on a surface or device area. With recent focus and attention being paid to cleanliness, disinfection, and sterilization, a need exists in the marketplace for efficient disinfection and/or sterilization of high-use items at shopping centers and food markets.

### BRIEF DESCRIPTION OF THE DRAWINGS

Aspects and many of the attendant advantages of the claims will become more readily appreciated as the same become better understood by reference to the following detailed description, when taken in conjunction with the accompanying drawings, wherein:

FIG. 1 shows an isometric diagram of a system for using ultra-violet light to sterilize shopping carts according to an embodiment of the subject matter disclosed herein;

FIG. 2 shows an interior view of the system for using ultraviolet light to sterilize shopping carts of FIG. 1 according to an embodiment of the subject matter disclosed herein;

FIG. 3 shows a plan view of the he system for using ultraviolet light to sterilize shopping carts of FIG. 1 according to an embodiment of the subject matter disclosed herein;

FIG. 4 shows a block diagram of the system of FIGS. 1-3 for using ultraviolet light to sterilize shopping carts according to an embodiment of the subject matter disclosed herein;

FIG. 5 shows steps of a method for sterilizing shopping carts using ultra-violet light according to an embodiment of the subject matter disclosed herein;

FIG. 6 shows an isometric diagram of another embodiment of a system for using ultra-violet light to sterilize shopping carts according to an embodiment of the subject matter disclosed herein;

FIG. 7 shows an exploded view of the system for using ultraviolet light to sterilize shopping carts of FIG. 6 according to another embodiment of the subject matter disclosed herein; and

FIG. 8 shows pictorial steps of a method for sterilizing shopping carts using ultra-violet light according to an embodiment of the subject matter disclosed herein.

### DETAILED DESCRIPTION

The following discussion is presented to enable a person skilled in the art to make and use the subject matter disclosed herein. The general principles described herein may be applied to embodiments and applications other than those detailed above without departing from the spirit and scope of the present detailed description. The present disclosure is not intended to be limited to the embodiments shown, but is to be accorded the widest scope consistent with the principles and features disclosed or suggested herein.

The subject matter disclosed herein is related to a system and method for disinfecting and/or sterilizing one or more high-touch implements, such as shopping carts, using ultraviolet (UV) light within a contained chamber for a specified period of time. In an embodiment, one or more shopping carts may be guided into a sterilization chamber through a front-side opening of an apparatus. Being at item of heavy use and repeated human contact, viruses and bacteria may collect and linger at high-touch areas, such as on shopping carts handles. Lingering bacterial load and viral load can lead to a reduction in overall health of a population by spreading communicable disease.

In a novel manner described herein, bacteria and viruses may be reduced or eradicated with sterilization and/or exposure to disinfectants on these high-touch regions of shopping carts. Disinfection does not necessarily kill all microorganisms, especially resistant bacterial spores and is less effective than sterilization, which is an extreme physical or chemical process that kills all types of life. Disinfectants work by destroying the cell wall of microbes or interfering with their metabolism. It is also a form of decontamination, and can be defined as the process whereby physical or chemical methods are used to reduce the amount of pathogenic microorganisms on a surface. However, sterilization may ensure that all bacterial microbes and viral loads are eliminated.

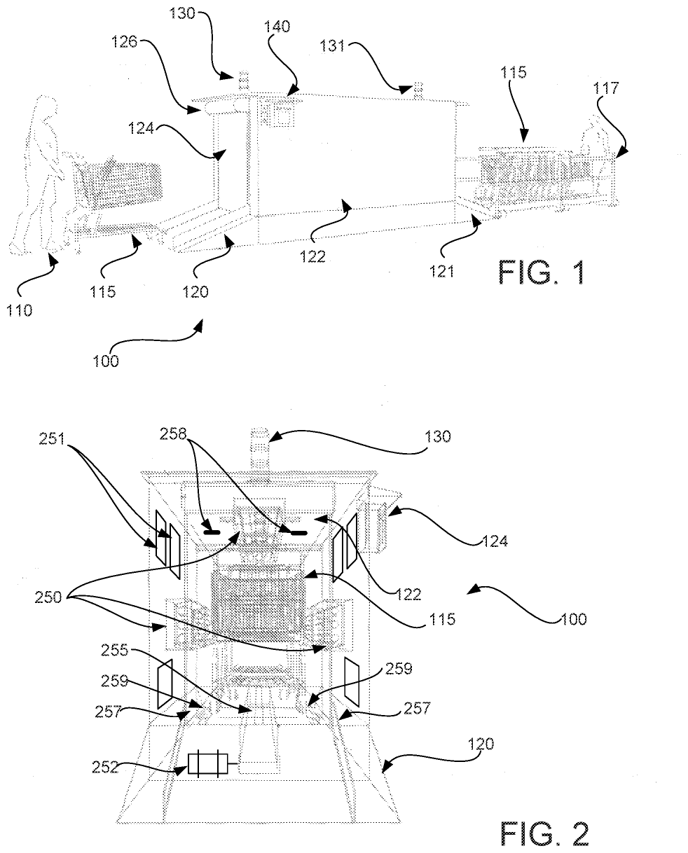

To this end, once the shopping cart is placed inside the sterilization chamber, a UV light process may be engaged whereby several UV lights will be turned on to flood the sterilization chamber with UV light. UV light has been shown to be effective at killing bacteria, viruses, mold, and other pathogens with exposure in as little as 5 seconds. In embodiments, the process may include exposures for longer time periods of time and intermittent periods of time. Additionally, some embodiments may deploy chemical disinfectants on high-touch surface areas to assist with the sterilization procedure. The sterilization procedures results in sterilizing a shopping cart for next use as sterilized carts may be collected in a corral designated form newly sterilized shopping intended for next use. These and other aspects and advantages are discussed below with respect to FIGS. 1-5.

FIG. 1 shows an isometric diagram of a system 100 for using UV light (and possibly additional disinfectants) to sterilize shopping carts 115 according to an embodiment of the subject matter disclosed herein. Within the system 100 of FIG. 1, a shopper or employee 110 may guide a shopping cart 115 in need of sterilization to an opening 124 of a sterilization chamber 122. Within the sterilization chamber 122, the system 100 may include several UV light banks (not shown in FIG. 1) configured to shine UV light from all angles toward any shopping cart 115 therein during a sterilization procedure. UV light is part of an ultraviolet light spectrum that is defined as light having a wavelength between 100-400 nm. Within the UV light spectrum is a subclassification of germicidal UV light that is typically characterized as having a wavelength of 185-280 nm. Germicidal UV light, because of its high frequency, is effective at killing bacteria, viruses, mold, and other pathogens within a few seconds of exposure. As such, if one were to use several UV lights within germicidal wavelengths limits to flood a sterilization chamber 122 having an item therein, bacteria, viruses, mold, and other pathogens may be eradicated (e.g., killed). If all bacteria, viruses, mold, and other pathogens are killed, then the item is considered sterilized.

In FIG. 1, the system 100 is shown having a sterilization chamber 122 surrounding by a plastic or metal shroud enclosure. The enclosure may be any suitable material (e.g., poly extruded plastics, aluminum, stainless steel, and the like) and may be decorated or branded to suit a company business interest. At the front-side (e.g., shopping cart entry side), a front-side opening 124 may be adorned with opaque or translucent roll-up door 126 or strip curtains (not shown in FIG. 1) such that a shopping cart 115 may easily push through the door opening or strip curtains. Further, the front-side of the sterilization chamber 122 may be affixed adjacent to an entry ramp 120 suited to facilitate easier entry of a shopping cart 115 in need of sterilization. The entry ramp 120 may be a flat, inclined platform suited to provide a path for an employee 110 to push a shopping cart 115 into the sterilization chamber 122 through the front-side opening 124. In other embodiments, entry ramp 120 may be a flat, inclined platform having wheel grooves to guide shopping cart wheels and suited to provide a path for an employee 110 to push and guide a shopping cart 115 into the sterilization chamber 122 through the front-side opening 124.

Once clear of the opening 124 (e.g., shopping cart is within the exposure chamber), the strip curtains or roll-up door 126 will revert to a default position of covering the opening 124. The strip curtains or roll-up door 126 may be opaque such that no germicidal UV lights escapes the sterilization chamber 122 or may be translucent with reflective material on the interior designed to keep as much germicidal UV lights within the sterilization chamber 122 as possible. Further, in other embodiments, the front-side covering 126 may be an actuated door or other covering that can be maneuvered or rotated to allow shopping carts 115 to access the interior of the sterilization chamber 122. Further, a roll-up door, actuated door, or strip curtain may be present at the exit although not shown in this view in FIG. 1. Additionally, each opening covering may be closed and locked when the sterilization chamber 122 is not open to the public or the system 100 is not intended to be in use.

The system 100 may further include a control panel 140 mounted to the exterior of the sterilization chamber 122 that enables a user to initiate a sterilization process. Such a control panel 140 may include hardware- or software-enabled functionality for starting, stopping, interrupting, or adjusting a sterilization process. The system 100 may include occupancy sensors (not shown), that may be thermal sensors placed in the interior of the sterilization chamber 122. that are meant to shut down the UV lights if a person enters the sterilization chamber 122 while the UV lights are on. The system 100 further includes one or more indicator lights (e.g., entry-side indicator light 130 and exit-side indicator light 131) to alert users to the internal sterilization process and/or stage of the cleaning process. In an embodiment, these occupancy sensors may be active when the indicator lights 130, 131 are turned on or may be enabled at the control panel 140 during a lighting sequence. In a further embodiment, the system 100 includes several differently-colored lights in a stack configuration within the entry-side indicator light 130 and exit-side indicator light 131. These indicator lights 130, 131 may indicate green (sterilization process is clear) or red (sterilization process is in progress) or other colors to indicate other stages of the sterilization process. Further, additional status lights on the top or sides of the enclosure may provide visual feedback of whether the system 100 is running with one or more carts in the sterilization chamber 122.

The system 100 includes an exit opening at the back-side of the enclosure. The exit opening may also have a roll-up door, actuated door, or strip curtains to serve the dual purpose of easy access for retrieving sterilized carts 115 as well as keeping germicidal UV light within the sterilization chamber 122. Further, the exit opening may be flanked by a shopping cart corral 117 for organizing sterilized shopping carts 115 that are available for customer use.

FIG. 2 shows an interior view of the system 100 for using UV lights 250 to sterilize shopping carts 115 of FIG. 1 according to an embodiment of the subject matter disclosed herein. In this view, one can see inside the sterilization chamber 122 within the enclosure. In this view, strip curtains and/or a roll-up door 124 are shown as transparent so that interior features can be seen. In specific, the interior may include shopping cart bumpers 257 at the left and rights sides inside the interior of the sterilization chamber 122 to assist in guiding the shopping carts 115 through the sterilization chamber 122. Further, along the interior sides of the sterilization chamber 122, the system 100 may include a motorized automated cart track 255 such that a shopping cart 115 may be maneuvered into the sterilization chamber 122 up to an engagement point, whereupon a motorized track 255 pulls the shopping cart 115 though the sterilization chamber 122 at a steady pace using an automated motor 252.

In further elaboration of the motorized track 255 for pulling shopping carts 115 through the sterilization chamber 122, the system 100 may include a pair of adjustable cart guides 259 inside that are configured to be adjusted to fit the width of the specific shopping carts 115 per the facility's shopping cart requirements. This feature allows for universal-fit systems and alleviates any need to customize a system for differently-sized shopping carts 115. The motorized mechanism 255 may pull the engaged shopping carts 115 through the sterilization chamber 122 and prevent any shopping carts 115 from slipping backwards. This motorized mechanism 255 is configured to engage shopping carts 115 without any modification or parts added to the shopping carts 115. The motorized mechanism 255 may be centrally located at the base of the sterilization chamber 122 as shown or may be mounted to one or both side walls of the sterilization chamber 122. The motorized mechanism 255 may include an electric or pneumatic motor 252 or any other motor capable of imparting linear motion to track system for maneuvering one or more shopping carts 115 through the sterilization chamber 122.

One can also see, in FIG. 2, three sets of germicidal UV lights 250 disposed on the interior of the sterilization chamber 122. In this embodiment, the UV lights 250 are permanently mounted to the left-side, right side, and top-side of the interior or the sterilization chamber 122. In other embodiments not shown, the UV lights 250 may be mounted at other locations inside the sterilization chamber 122 including the bottom and in all four corners. In some embodiments, the UV lights 250 may be disposed at all angles within the sterilization chamber 122. In still further embodiments, the UV lights 250 may be mounted in a movable manner (e.g., rotatable manner) within the sterilization chamber 122 such that during a sterilization process, the set of movably mounted UV lights 250 rotate about the interior of the sterilization chamber 122 to ensure exposure of germicidal UV light to all surfaces of a shopping cart 115. Further yet, in an effort to increase the amount of UV light that floods the sterilization chamber 122, several reflectors 251 may be disposed on the interior walls of the sterilization chamber. Although a discrete number of reflectors 251 are shown in FIG. 2, any number may be disposed inside the chamber including every interior surface having some manner of reflectivity inside the sterilization chamber 122.

The system 100 may include occupancy sensors 258, that may be thermal sensors placed in the interior of the sterilization chamber 122. that are meant to shut down the UV lights 250 if a person enters the sterilization chamber 122 while the UV lights 250 are on. As described previously, the system 100 further includes one or more indicator lights 130 to alert users to the internal sterilization process and/or stage of the cleaning process. In an embodiment, these occupancy sensors 258 may be active when the indicator lights 130 are turned on or may be enabled at the control panel 140 during a lighting sequence.

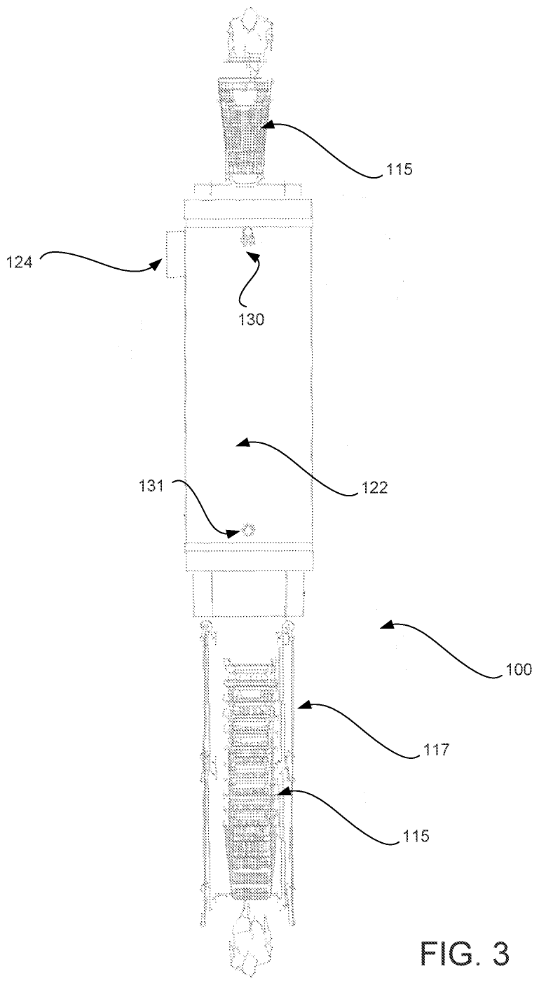

FIG. 3 shows a plan view of the system 100 for using UV light to sterilize shopping carts 115 of FIG. 1 according to an embodiment of the subject matter disclosed herein. From this perspective, one can see that several shopping carts 115 may be within the sterilization chamber 122 during a sterilization procedure. The interior of the sterilization chamber 122 may be lined with highly reflective surface so as to illuminate the entire chamber with germicidal UV light while the shopping cart sterilization procedure in undertaken. Further, the sterilization process may be an ongoing process such that shopping carts 115 may be pushed, by a user, into the front-end opening 124 of the sterilization chamber 122 until each shopping cart 115 engages the motorized mechanism 255 mounted inside the sterilization chamber 122. The motorized mechanism 255 may then pull the shopping carts 115 though the sterilization chamber 122 whereby germicidal UV light is consistently or intermittently shone within the sterilization chamber 122. As the shopping cart 115 is pulled through, it may exit the sterilization chamber 122 at a back-end opening into a shopping cart corral 117 having guidance tracks and bumpers for space-efficient stacking of shopping carts 115 in a nested manner.

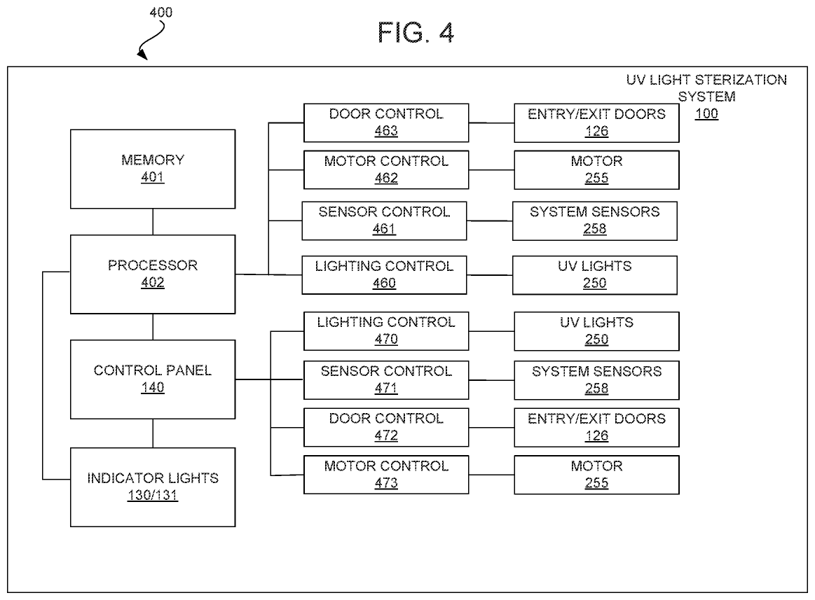

FIG. 4 shows a block diagram 400 of the system 100 of FIGS. 1-3 for using UV light to sterilize shopping carts according to an embodiment of the subject matter disclosed herein. The system 100 may include a processor 402 that is capable of executing non-transitory computer-readable instructions that may be stored in a memory 401 coupled to the processor 402. Further, the processor 402 may be configured to receive inputs from a communicatively coupled control panel 140 that an operator can use. The control panel 140 may include software-enabled or hardware-enabled push-button inputs as well as display-based indicators. Further, specific lighting indicators 130/131 may be coupled to the control panel 140 and coupled to the processor 402 to indicate to an observer basic operating conditions of the system 100. In this manner, the system 100 may have the ability to engage in fully automated control through programmatic instruction as executed by the processor 402 or manual control as executed through the control panel 140 by an operator/user.

As such, the system 100 may include an automated lighting control interface 461 for controlling one or more germicidal UV lights 250 under the direction or the processor 402 in an automated operating procedure. Further, the system 100 may include an automated door control interface 461 for controlling the entry and exits doors under the direction or the processor 402 in an automated operating procedure. Further yet, the system 100 may include a manual lighting control interface 471 for controlling one or more germicidal UV lights 250 under the direction or the control panel 140 in a manual operating procedure. Further, the system 100 may include a manual lighting control interface 471 for controlling one or more germicidal UV lights 250 under the direction or the control panel 140 in a manual operating procedure. The system 100 may include a motor control interface 480 for controlling one or more mechanized trams 255 via the motor 252 for the automated cart motion track under the direction or the processor 402.

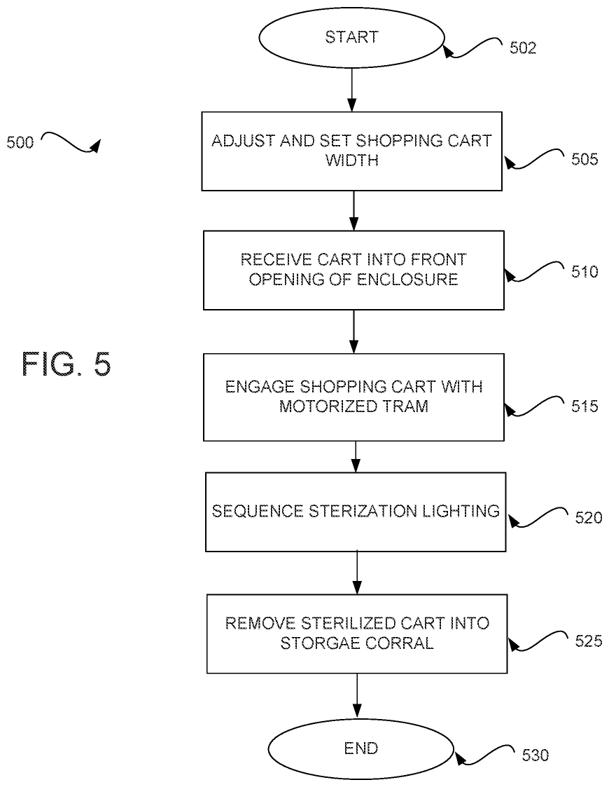

FIG. 5 shows steps of a method 500 for sterilizing shopping carts 155 using UV light according to an embodiment of the subject matter disclosed herein. The method may begin at a start step 502 and then proceed to initial steps for be ready to receiving a first shopping cart into and through the front-side opening of the sterilization chamber 122 of FIGS. 1-3). As a preliminary step to receiving shopping carts 155 to be sterilized, a technician may adjust the width of the guide tracks within the sterilization chamber, at step 505, to suit the specific design of the shopping carts at the establishment that owns the shopping carts. Having set the guide track width, the sterilization chamber is ready to receive one or more shopping carts to the interior for a sterilization procedure.

At step 510, a human user may push one or more shopping carts to the front-side opening of the sterilization chamber 122 to the point in which the automated motorized tram can engage the first of the shopping carts. Once the first cart is far enough into the front-opening, the shopping cart may engage the motorized tram and begin moving under the power and direction of the motorized tram system at step 515. When the set of shopping carts is fully within the sterilization chamber 122, a leading edge of the set of shopping carts may actuate a threshold sensor that causes initiation of the germicidal UV light procedure. In further embodiments, an additional trailing edge threshold sensor may need to be actuated at the same time so that a stack of shopping carts may undergo the procedure all at once. That is, a stack of shopping carts will have a sufficient length that is greater than one or two shopping carts such that a leading-edge sensor is tripped at the same time as a trailing-edge sensor—which indicates that a sufficient number of shopping carts is in the chamber to begin the sterilization procedure. In other embodiments, this step may be triggered by a weight sensor wherein a threshold of shopping carts has been positioned inside the sterilization chamber 122 to trigger the sterilization procedure (e.g., a weight equal to 10 shopping carts, for example).

Once triggered, the sterilization procedure may begin at step 520. Any door need in need of closing at the entry-side or exit-side is actuated as the procedure begins. Once safety protocols have been met, the germicidal UV lights may be sequenced. The germicidal UV lighting sequence then sterilizes the shopping cart within the sterilization chamber 122 through a series of on-off cycles and possible light carriage rotations for a requisite time. One such requisite time may be 8-10 seconds of high-level exposure. Other sequences may involve longer periods of time with lower-level exposures or with different sets of lights being sequenced through a pattern. Once the specific procedure of sequenced lighting occurs, the shopping carts inside the sterilization chamber 122 are considered sterilized and the shopping cart may be removed from the sterilization chamber 122 at an exit opening that collects the sterilized shopping carts into a corral before at step 525. The method then finishes at an end step 530.

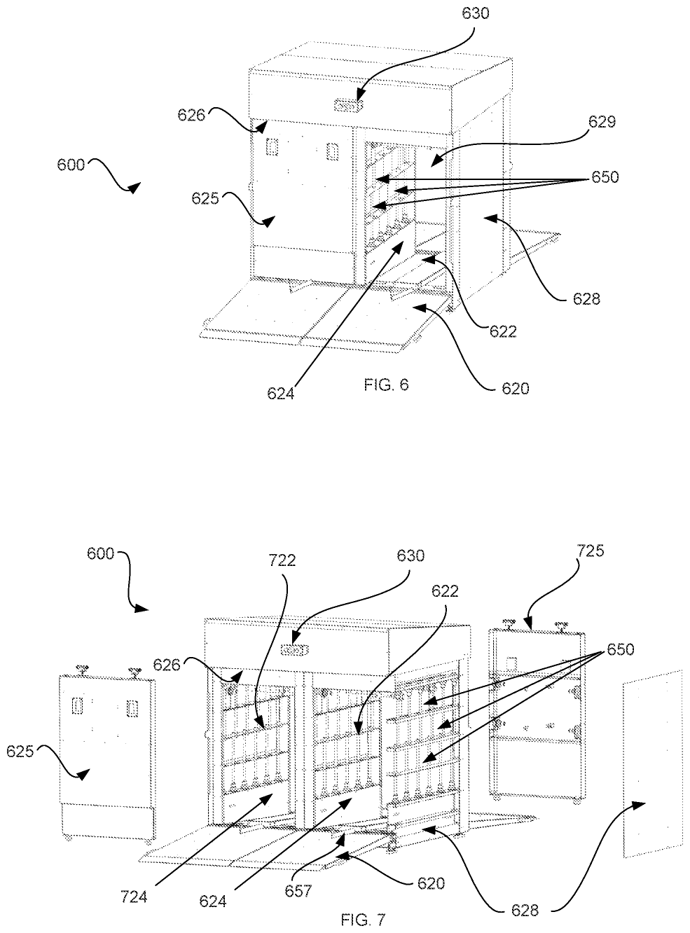

FIG. 6 shows an isometric diagram of another embodiment of a system 600 for using ultra-violet light to sterilize shopping carts according to an embodiment of the subject matter disclosed herein In FIG. 6, the system 600 is shown having a sterilization chamber 622 surrounding by a plastic or metal shroud enclosure 628. The enclosure may be any suitable material (e.g., poly extruded plastics, aluminum, stainless steel, and the like) and may be decorated or branded to suit a company business interest. At the front-side (e.g., shopping cart entry side), a front-side opening 624 may be adorned with an opaque door 625 on a sliding actuator 626. Further, the front-side of the sterilization chamber 622 may be affixed adjacent to an entry ramp 620 suited to facilitate easier entry of a shopping cart (not shown in this embodiment) in need of sterilization. The entry ramp 120 may be a flat, inclined platform suited to provide a path for an employee to push a shopping cart into the sterilization chamber 622 through the front-side opening 624. In other embodiments, entry ramp 620 may be a flat, inclined platform having wheel grooves to guide shopping cart wheels and suited to provide a path for an employee to push and guide a shopping cart into the sterilization chamber 622 through the front-side opening 624. Further yet, the ramp 620 may also be rotatably attached to the based on the enclosure 628 such that the ramp may fold up for storage and/or transport.

Once clear of the opening 624 (e.g., shopping cart is within the sterilization chamber 622), the sliding door 125 may be actuated to slide right to cover the opening 624 and lock to a default position. A similar rear door (not shown) may also be actuated in a similar manner to cover the rear opening 629. The interior of the sterilization chamber 622 includes several germicidal UV lights 650 mounted in a manner to flood the entire chamber with germicidal UV light. The doors 626 may be opaque such that no germicidal UV light escapes the sterilization chamber 622 or may be adorned with reflective material on the interior designed to keep as much germicidal UV light within the sterilization chamber 622 as possible.

The system 600 may further include a control panel (not shown in FIG. 6) mounted to the exterior of the sterilization chamber 622 that enables a user to initiate a sterilization process. Such a control panel may include hardware- or software-enabled functionality for starting, stopping, interrupting, or adjusting a sterilization process. The system 600 may include occupancy sensors (not shown), that may be thermal sensors placed in the interior of the sterilization chamber 622. that are meant to shut down the UV lights if a person enters the sterilization chamber 622 while the UV lights are on. The system 600 further includes one or more indicator lights 630 to alert users to the internal sterilization process and/or stage of the cleaning process. In an embodiment, these occupancy sensors may be active when the indicator lights 630 are turned on or may be enabled at the control panel during a lighting sequence.

The system 600 includes an exit opening 629 at the back-side of the enclosure 628. The exit opening may also have a roll-up door, actuated door, or strip curtains to serve the dual purpose of easy access for retrieving sterilized carts as well as keeping germicidal UV light within the sterilization chamber 622. Further, the exit opening may be flanked by a shopping cart corral (not shown in FIG. 6) for organizing sterilized shopping carts that are available for customer use.

FIG. 7 shows an exploded view of the system 600 for using UV lights 650 to sterilize shopping carts of FIG. 6 according to an embodiment of the subject matter disclosed herein. In this view, one can see inside the opening 624 into the sterilization chamber 622 within the enclosure 628. Further, one can now see into a second opening 724 leading to a second sterilization chamber 722 side-by-side with the first sterilization chamber 622. Further yet, the interior may include shopping cart bumpers and or adjustable cart guides 657 the interiors of the sterilization chambers 622, 722 to assist in guiding the shopping carts through the respective sterilization chambers 622, 722. Having two side-by-side sterilization chambers 622, 722 allows the doors 625, 725 to slide back and forth to fully enclose one side or the other for a sterilization procedure while a newly sterilized shopping may be retrieved from the now open sterilization chamber such that the now empty sterilization chamber is ready for another shopping cart to sterilize.

One can also see, in FIG. 7, nine sets of germicidal UV lights 650 disposed on the interior of the first sterilization chamber 622 as well as another nine sets of germicidal UV lights 650 disposed on the interior of the second sterilization chamber 722 In this embodiment, the UV lights 650 are permanently mounted to the left-side, right side, top-side, front and rear of the interior or the sterilization chambers 622, 722. In other embodiments not shown, the UV lights 650 may be mounted at other locations inside the sterilization chambers 622, 722 including the bottom and in all four corners. In some embodiments, the UV lights 650 may be disposed at all angles within the sterilization chambers 622, 722.

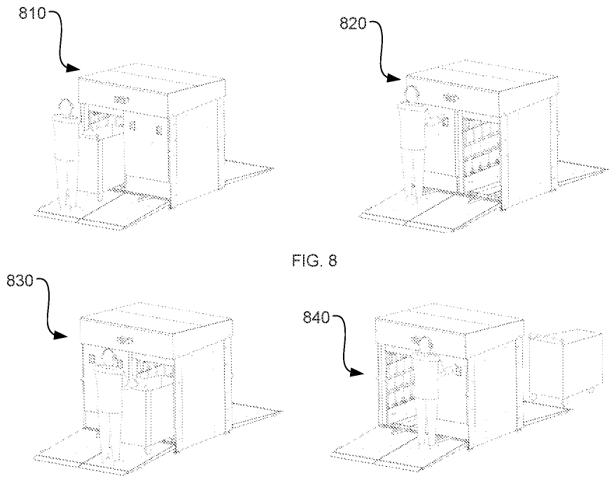

FIG. 8 shows pictorial steps of a method for sterilizing shopping carts using ultra-violet light according to an embodiment of the subject matter disclosed herein. In this figure, the process by which an operator may utilize the side-by-side sterilization chambers in tandem is shown in a series 810-840 of pictorials. In a first pictorial 810, an operator may load a cart into the left-side sterilization chamber in that the doors are enclosing the right-side sterilization chamber. Thus, a shopping cart in need of sterilization may be loaded. Then, in a second pictorial 820, the operator may cause the doors to slide to the left to enclose the left-side sterilization chamber such that the chamber may engage a lighting sequence to sterilize the loaded shopping cart. In some embodiments, when the doors slide all the way to one side, the enclosed sterilization chamber automatically engages a lighting procedure. Then, in a third pictorial 830, while the shopping cart is undergoing the sterilization procedure in the left-side, an operator may load a cart into the right-side sterilization chamber in that the doors are enclosing the left-side sterilization chamber. Thus, a shopping cart in need of sterilization may be loaded here now. Then, in a fourth pictorial 840, the operator may cause the doors to slide back to the right to once again enclose the right-side sterilization chamber such that the chamber may engage a lighting sequence to sterilize the other loaded shopping cart.

All references, including publications, patent applications, and patents, cited herein are hereby incorporated by reference to the same extent as if each reference were individually and specifically indicated to be incorporated by reference and/or were set forth in its entirety herein.

The use of the terms “a” and “an” and “the” and similar referents in the specification and in the following claims are to be construed to cover both the singular and the plural, unless otherwise indicated herein or clearly contradicted by context. The terms “having,” “including,” “containing” and similar referents in the specification and in the following claims are to be construed as open-ended terms (e.g., meaning “including, but not limited to,”) unless otherwise noted. Recitation of ranges of values herein are merely intended to serve as a shorthand method of referring individually to each separate value inclusively falling within the range, unless otherwise indicated herein, and each separate value is incorporated into the specification as if it were individually recited herein. All methods described herein can be performed in any suitable order unless otherwise indicated herein or clearly contradicted by context. The use of any and all examples, or exemplary language (e.g., “such as”) provided herein, is intended merely to better illuminate embodiments and does not pose a limitation to the scope of the disclosure unless otherwise claimed. No language in the specification should be construed as indicating any non-claimed element as essential to each embodiment of the present disclosure.

Different arrangements of the components depicted in the drawings or described above, as well as components and steps not shown or described are possible. Similarly, some features and sub-combinations are useful and may be employed without reference to other features and sub-combinations. Embodiments have been described for illustrative and not restrictive purposes, and alternative embodiments will become apparent to readers of this patent. Accordingly, the present subject matter is not limited to the embodiments described above or depicted in the drawings, and various embodiments and modifications can be made without departing from the scope of the claims below.

What is claimed is:

1. A sterilization device, comprising:
an enclosable chamber having a first opening and a second opening;
a guide disposed inside the chamber configured to guide a shopping cart from the first opening to the second opening;
a plurality of ultraviolet light disposed on the inside of the chamber and configured to shine light on the shopping cart when guided through the chamber; and
a control system configured to receive at least one control signal from a sensor disposed on the inside of the chamber, the control system controlling the plurality of ultraviolet lights in response to the at least one control signal.

2. The sterilization device of claim 1, further comprising:
a first door covering configured to retractably enclose the chamber by covering the first opening; and
a second door covering configured to retractably enclose the chamber by covering the seconding opening.

3. The sterilization device of claim 2, further comprising a second sensor disposed in the chamber and configured to actuate the first and second door coverings in response to detecting the shopping cart.

4. The sterilization device of claim 1, wherein the sensor further comprises a motion detection sensor configured to detect when the shopping cart breaches a plane within the chamber.

5. The sterilization device of claim 1, wherein the sensor further comprises a weight detection sensor configured to detect when weight from the shopping cart exceeds a threshold weight of the sensor.

6. The sterilization device of claim 1, wherein the plurality of ultraviolet lights further comprises germicidal ultraviolet lights configured to produce light having a bandwidth between 185-280 nm.

7. The sterilization device of claim 1, wherein the guide further comprises a pair of guide rails disposed in the chamber and spaced apart from each other at a width to match a width of wheels on the shopping cart.

8. The sterilization device of claim 9, wherein the guide rails further comprise adjustable-width guide rails configurable to be spaced apart from each other at a width to match a width of wheels on a different shopping cart.
